(19) United States
(12) Patent Application Publication
(10) Pub. No.: US 2019/0189321 A1
(43) Pub. Date: Jun. 20, 2019

(54) ELECTROMAGNETIC DEVICE AND MANUFACTURING METHOD THEREOF

(71) Applicant: ELTEK S.P.A., Casale Monferrato (AL) (IT)

(72) Inventors: Fabrizio Bardi, Vignale Monferrato (IT); Mauro Zorzetto, Casale Monferrato (IT)

(21) Appl. No.: 16/301,395

(22) PCT Filed: May 3, 2017
(86) PCT No.: PCT/IB2017/052556

§ 371 Date: Nov. 13, 2018

(30) Foreign Application Priority Data
May 13, 2016 (IT) 102016000049478

(51) Int. Cl. H01F 5/04; A61K 49/18; A61K 49/22; B22F 1/02; B22F 9/24; F02M 51/00; H01F 41/10; H01F 7/06

(52) U.S. Cl. CPC H01F 5/04; A61K 49/183; A61K 49/1866; A61K 49/225; B22F 1/02; H01F 2007/062; F02M 51/005; H01F 41/10; H01F 7/06; C01P 2004/34; B22F 9/24

(57) ABSTRACT

An electromagnetic device, such as an actuator for electrovalves or injectors, particularly for endothermal engines includes a coil winding of an electric wire covered with an insulating layer made of overmoulded plastic material. In order to prevent the electric wire from breaking at the ends thereof connected to the electric terminals, a protective insert is applied prior to the plastic moulding step. The insert is also made of thermoplastic material, so that it can melt locally together with the cover material, thereby ensuring a stable and effective union during the moulding process. The invention also relates to a process for manufacturing the device.

### BACKGROUND OF THE INVENTION

### 1. The Field of the Invention

From a general viewpoint, the present invention relates to an electromagnetic device comprising at least one wound electric wire, such as a solenoid and/or an electromagnet and/or an electromagnetic coil, or the winding of an antenna and/or an inductor for an electronic circuit and/or the winding of an electric actuator or motor.

### 2. The Relevant Technology

Preferably, the device according to the invention belongs to an electromagnetic actuator of an electrovalve, particularly for apparatus or systems for injecting a fluid, e.g., an apparatus or system for injecting fuel and/or additive for combustion or endothermal engines and/or vehicles. The present invention also relates to at least one method or process for manufacturing and/or assembling said device.

As is known, some electromagnetic devices comprise a body or reel whereon an electrically conductive wire is wound, typically a copper wire covered with electrically insulating material, such as enamel or resin, e.g., polyurethanes, polyestherimide, polyamide-imide, polymide.

To the reel, electric terminals are typically fastened for creating an electric connection, hereafter also referred to, for brevity, simply as “terminals”; said terminals are typically provided with shaped portions useful for establishing a mechanical and electrical connection with the ends of the electrically conductive wire or conductor, hereafter also referred to, for brevity, simply as “wire”.

The ends of the wire can be fastened to the electric terminals by mechanical deformation of portions of the terminal itself and/or by soldering, e.g., electric soldering and/or soldering by addition of soldering material or metal alloy. In order to allow the wire to be wound on the reel and to be fixed to the electric terminals by automatic machines, the electric terminals include portions acting as fastening points for the electric wire, which extend in a direction substantially perpendicular to a longitudinal axis of the winding of the coil and/or solenoid.

In particular, the terminals provide some clearance around said fastening portion to allow the wire to be wound, in particular by means of a wire guiding element moving around said fastening portion.

Some examples of electromagnetic windings mechanically and electrically fastened to fastening portions of electric terminals in accordance with the above explanation have been known for decades, as demonstrated by German patent publication no. DE3615806 and utility model publication no. DE8700157U, to which reference should be made for further details.

In such known devices, after the conductive wire has been wound on the reel and the ends of the wire have been fastened to the electric terminals, a protective body or layer is added to cover the winding and at least part of the electric terminals, e.g., an overmoulded insulating protective covering body made of a plastic, thermoplastic, polymer or resin material.

Some examples of overmoulding of electromagnetic windings in accordance with the above explanation are known in the art, such as those described in International patent application WO 01/96086 and European patent application EP1614518, to which reference should be made for further details.

In other known devices, the protective covering body of the electric wire and/or solenoid and/or terminals forms at least part of the body of an electromagnetic device, such as a fuel injector; for an example of this state of the art, reference should be made to United States patents or applications U.S. Pat. No. 5,185,919, US2007/0290447, U.S. Pat. No. 5,823,445.

The process for manufacturing the electromagnetic device referred to herein substantially requires the presence of a clearance zone around the fastening portion for fastening the electric wire to the corresponding terminal, in particular a clearance zone between the body or reel whereon the wire is wound and said fastening portion of the terminal, in order to allow winding at least a part of an end of the electric wire.

Usually the process employed for manufacturing electromagnetic devices known in the art is carried out by an automatic apparatus fitted with at least one mobile wire guiding element of the type adapted to move along predefined paths under the control of an electronic control system; during the process, the reel with the terminals and the wound wire is then covered or overmoulded, at least partly, with a polymer, such as a thermoplastic material or a thermosetting material or a resin.

The known product thus obtained, i.e., the electromagnetic device comprising a coil or winding, is not however exempt from reliability risks that may impair the function it is intended for, particularly when the wire size is small or capillary, as is the case of certain coils for injectors to be used in engines or vehicles.

In fact, the present Applicant observed that, at least as concerns coils with an electric wire size smaller than 0.2 mm, particularly of the type with a wire diameter in the range of 0.15 mm to 0.16 mm, during the cover moulding or overmoulding operations mechanical stress arises which may cause the electric wire to break, leading to a large number of discarded parts; furthermore, such mechanical stress may cause latent damage that may not be detected during the production process, revealing itself only after some time in products already sold. In particular, this may occur in case of use of the product in extreme conditions, such as the use in a combustion engine or vehicle, or in the presence of vibrations and/or wide temperature variations, which also cause thermal expansion and/or contraction in the coil materials. The Applicant also verified that this risk is even higher in electromagnetic devices to be used in critical conditions, such as electromagnetic devices installed in vehicles and/or vehicle engines and/or endothermal engines in general, which are subject to high thermal stress and/or mechanical stress, such as vibration, acceleration, shocks and the like.

It was also verified that said risk condition is more frequent when the electromagnetic device is applied to at least one electrovalve of a line or apparatus for injecting fuel or additive into an endothermal engine and/or an exhaust system and/or an emission control system, or anyway in environmental conditions wherein the device is subject to wide temperature ranges, e.g., when an engine is started in cold weather conditions, which may be below 0° C. (e.g., as low as −40° C.), followed by fast heating to temperatures in excess of 100° C. (e.g., up to +125° C.), and then the engine is turned off and cools down again (−40° C.). Let us consider, for example, an endothermal engine of a vehicle or an electric generator or an air compressor exposed to winter or arctic environmental conditions.

Such temperature changes cause mechanical stress, also referred to as “thermal stress” or “thermal shock”, adding to the mechanical stress induced by the engine vibration: all these stresses will be transmitted, whether directly or indirectly through other parts, to said electrovalve for injecting or feeding fuel or additive into the engine and/or exhaust system.

The Applicant also verified, surprisingly, that such mechanical stress and the resulting risk of failure of the electric wire grows higher in those areas where the protection covering or overmoulding is thicker, particularly in that wire portion which crosses at least partly the protective cover that fills said clearance zone for winding the wire around the electric terminal, such as the covering or overmoulding part in the clearance zone between the reel and the wire fastening portion.

The Applicant originally thought that, in order to improve the state of the art, such risks of malfunction can advantageously be eliminated or reduced and/or the performance of the aforesaid electromagnetic devices can be improved.

A general technical problem at the basis of the present invention is therefore to overcome the above-described problems of the prior art.

A further technical problem addressed herein is to make the operation of the electromagnetic device reliable, independently of the production conditions and/or of the apparatus with which it may be associated, i.e., regardless of the conditions of use.

Another technical problem addressed herein is to provide a device which proves to be reliable even when covering or overmoulding materials are used which are characterized by relatively high shrinkage, e.g., during the moulding process in the production cycle, and/or by high thermal expansion and/or contraction, e.g., in operating conditions.

A general idea that solves the above-mentioned technical problems is to improve the safety and/or reliability and/or performance of electromagnetic devices, in particular of devices such as electromagnetic valves and/or actuators and/or motors, or anyway of devices including electric windings, such as a solenoid and/or an electromagnet and/or an electromagnetic coil.

According to a further aspect, another solution proposed by the invention is to improve the safety and/or reliability and/or performance of a coil and/or an electrovalve for apparatus or systems for injecting a fluid, such as an apparatus or system for injecting fuel and/or additive for combustion or endothermal engines and/or vehicles.

According to a further, more specific aspect, the invention aims at providing an electromagnetic device of the type considered above, which has such structural and/or functional characteristics as to prevent or reduce the risk of failure of the electric wire of an electromagnetic device.

The invention also comprises a method or a process for manufacturing and/or assembling an electromagnetic device, such as a coil and/or an electrovalve and/or an actuator, or the like, for apparatus in general, and in particular for apparatus for injecting fuel or additive into internal combustion or endothermal engines and/or exhaust systems and/or emission control systems.

In order to implement this solution to the above-mentioned technical problem, the invention teaches to arrange additional or auxiliary means or elements or inserts, for supporting and/or protecting the electric wire and/or for reducing the thickness of the covering material, along at least part of the path followed by the wire, preferably in the areas subject to the risk of damage, such as said clearance zone between the reel and the fastening portion where the electric wire is secured to the electric terminal; said means or elements or inserts will hereafter also be referred to as “additional means” or “additional inserts” or simply as “inserts”.

One inventive idea is to arrange additional means for supporting that section of electric wire which extends in the areas subject to risk of breakage or damage, such as said clearance zone, in particular between the reel and the fastening portion where the electric wire is secured to the electric terminal, preferably for the purpose of creating support zones for the wire between the reel and the electric terminal.

A further inventive idea is to arrange additional means for protecting that section of the electric wire which extends through areas subject to the risk of breakage or damage, such as said clearance zone, i.e., between the reel and the fastening portion where the electric wire is secured to the electric terminal, particularly for the purpose of protecting the electric wire against the thrust exerted by the covering material during the moulding process steps, preferably carried out by injecting a polymer or a thermoplastic or thermosetting material or a resin into a mould, into which the reel has been previously inserted, with the electric wire wound thereon and secured to the electric terminals. To this end, the mould is preferably so designed that the material flow is injected into predefined points and arrives at the electric wire that needs to be protected and/or at the clearance zone from such a direction that the electric wire will be shielded or protected against the flow, or anyway with a predefined flow in a direction that will optimize the protection function of said additional means, such as a flow at least partly shielded or diverted by at least one wall of the additional means.

Preferably, additional means are used which can support the wire in such a way as to counter the thrust exerted by the flow of moulded material and/or which can protect the wire by diverting the flow of moulded material.

The provision of additional means for protecting or shielding and/or supporting the wire facilitates the moulding, while possibly also allowing the use of higher pressure and/or speed for injecting and diffusing the fluidic polymeric material into the mould, particularly for the purpose of reducing the moulding cycle time, thus reducing the cost of the device 1.

Another inventive idea is to arrange additional means for reducing the thickness of the covering or overmoulding material in the areas subject to risk of breakage or damage, such as said clearance zone, in particular between the reel and the fastening portion where the electric wire is secured to the electric terminal, preferably for the purpose of creating a thinner covering or overmoulding layer in the areas crossed or occupied by the electric wire between the reel and the electric terminal.

Advantageously, additional supporting and/or protective and/or thickness-reducing means are preferably arranged, at least partly, between the reel or inductive winding and portions for fastening or connecting the wire to the electric terminals.

In accordance with a particular solution to the technical problem, the additional means are fixed or coupled, at least partly, to at least one of the reel and at least one electric terminal, preferably for the purpose of at least facilitating the handling operations during the production cycle; to this end, coupling and/or engaging means are provided, which are at least partly formed on the additional means and/or at least partly formed on the reel and/or the terminals, such as coupling and/or engaging means operating by interference and/or hooks and/or rises and/or seats.

In accordance with a further solution to the technical problem, the additional means are provided with fixing elements to be secured to the covering body, for fixing or positioning the additional means by means of the covering material and/or by injecting or overmoulding the protection material onto at least one of the reel and at least one electric terminal, particularly at finished device level. Said fixing elements are, for example, rises or seats covered or filled, at least partly, with the covering or overmoulding material, such as lateral reliefs in the form of inclined planes or steps.

According to one inventive aspect, the electromagnetic device comprises or is fitted with an additional or auxiliary element made from polymer, such as a polyamide (PA), particularly of the PA6 and/or PA66 types, or a polybutylenterephthalate (PBT) or a polyphenylensulfide (PPS), preferably with the addition of a reinforcement additive or filler, such as fiberglass, e.g., in a quantity of 35%.

According to a further inventive aspect, the additional element is made from a polymer suitable for mechanically and/or chemically binding to the covering or overmoulding material, preferably by mutual melting or bonding during the overmoulding process; preferably, the additional element and the covering comprise at least one of a polyamide (PA, PA6, PA66) or a polybutylenterephthalate (PBT) or a polyphenylensulfide (PPS) or a thermosetting polymer, such as an epoxy resin, preferably with the addition of an additive or filler, such as fiberglass, e.g., in a quantity of 35%.

According to a further inventive aspect, the covering or overmoulding material is adapted to promote adhesion and/or chemical binding (such as a covalent and/or ionic or ionic-bridge bond) to the material of the additional element and/or the reel, or between the covering material and the material of the additional element and/or the reel an adhesion promoting material or substance (primer) is interposed, which creates a thin intermediate layer, preferably having a thickness of 1 microns to 20 microns, suitable for causing said materials or parts of the device to adhere and/or bind chemically to each other.

The features of the invention are more specifically set out in the claims appended hereto, which are an integral part of the description and/or of the technical teaching of the invention.

### BRIEF DESCRIPTION OF THE DRAWINGS

Such features, the effects resulting therefrom and/or the advantages of the invention will become more apparent in the light of the following description, in which reference will be made to the annexed drawings, which are supplied by way of illustrative and non-limiting example, wherein:

FIGS. 1 and 2 are axonometric views of an electromagnetic device according to the invention, in respective conditions turned by 180° relative to a longitudinal axis;

FIGS. 3 and 4 are, respectively, a top view and a front view of the device of FIGS. 1 and 2;

FIGS. 5 and 6 are, respectively, a bottom view and a side view of the device of the preceding figures;

FIGS. 7 and 8 are axonometric views of the above device with a part thereof removed, in respective conditions turned by 180°;

FIGS. 9 and 10 are views from respective angles of the above device and an additional means or insert according to the invention;

FIGS. 11 and 12 are axonometric views from different angles of some distinct parts of the device according to the invention;

FIGS. 13 and 14 are axonometric views from respective angles of a first example of an additional means or insert of the device of the preceding figures;

FIGS. 15 and 16 are axonometric views from different angles of a detail of the device according to the invention;

FIGS. 17 and 18 are axonometric views from different angles of a detail of a second example of the device according to the invention;

FIG. 19 schematically shows some steps (A), (B), (C) of the process for making the device of the preceding figures;

FIGS. 20, 21 and 22 are magnified views of respective parts of the device of the preceding figures;

FIGS. 23 and 24 show further magnified views from different angles, with a part removed, of a detail of the device according to the invention;

FIGS. 25 and 26 schematically shows respective steps of the process for making the device of the preceding figures.

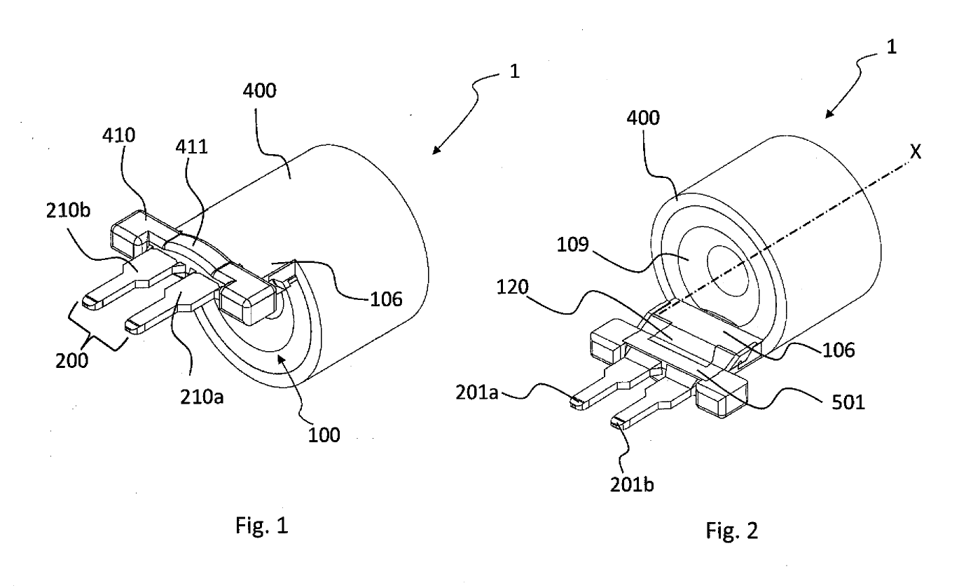

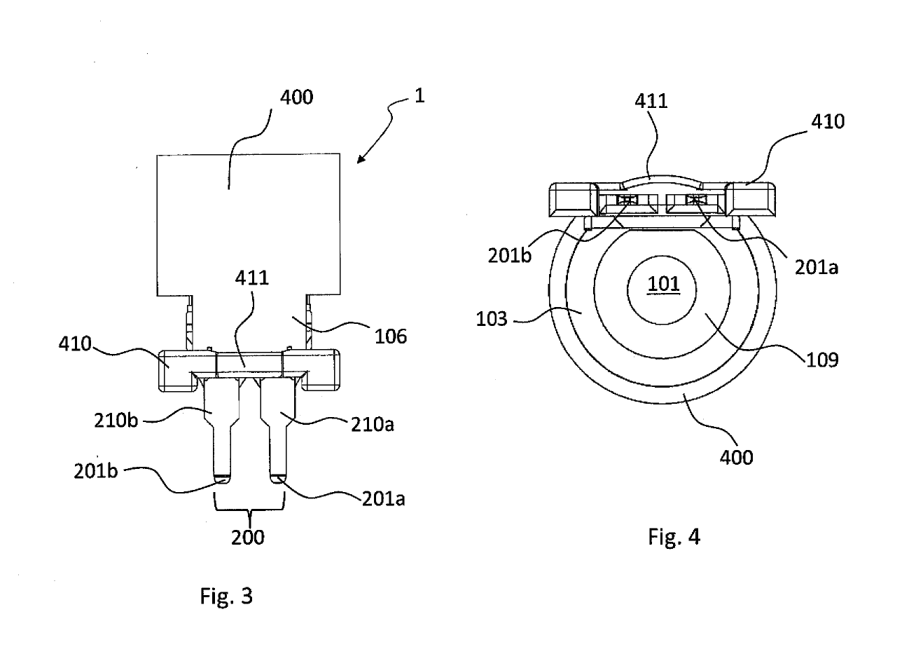

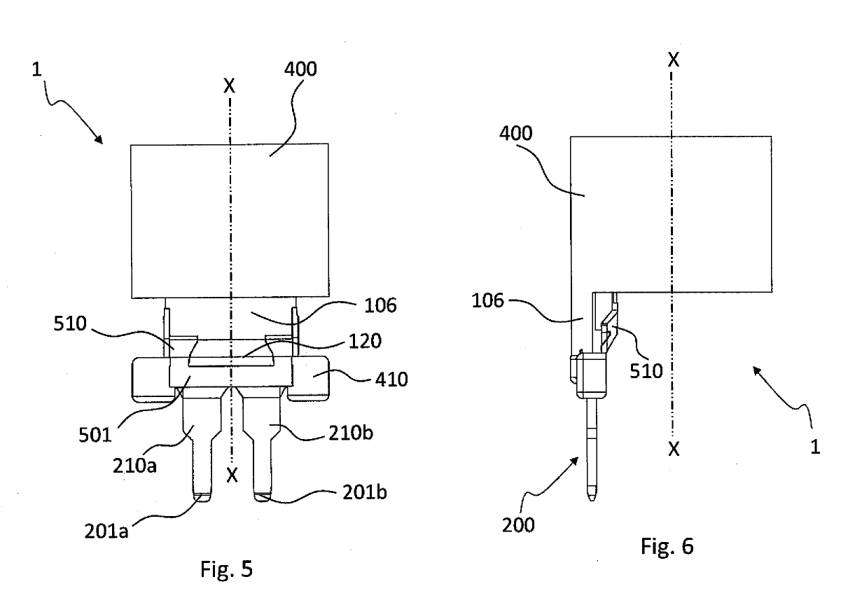

### DETAILED DESCRIPTION OF THE PREFERRED EMBODIMENTS

Before proceeding any further with a detailed description, it must be pointed out that particular configurations, structures or features described herein as non-limiting examples may be considered individually or combined as deemed appropriate in one or more embodiments, even different from those exemplified herein. Also, the references below are therefore used only for simplicity's sake and do not limit the protection scope or extent of the various embodiments. Any numerical and spatial references and/or definitions such as “upper”, “lower”, “on”, “under”, “high”, “low” refer to the exemplary drawings and should not be understood as limitations.

With reference to the drawings, 1 designates as a whole a preferred example of a device according to the invention, such as an electromagnetic device comprising at least one wound electric wire, in particular a solenoid or an electromagnet or an electromagnetic coil, preferably being a part of an electromagnetic actuator of an electrovalve or an injector, in particular intended for use in apparatus or systems for injecting fuel or additive into combustion or endothermal engines of vehicles and/or exhaust systems and/or emission control systems.

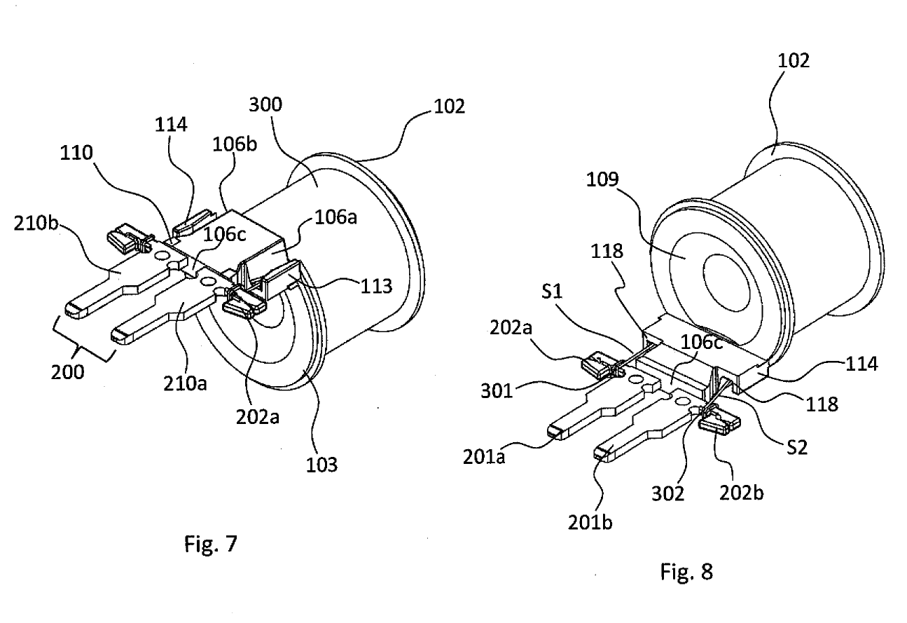

The device 1 according to the invention comprises at least one element or reel 100 for winding and/or supporting an electric wire, wherein a central or support body 101, typically cylindrical and axially hollow, is delimited at its ends by two walls or flanges 102 and 103, i.e., a rear first one and a front second one with reference to the drawings (FIGS. 7 and 8).

Alongside at least the front flange 103 of the reel 100, such as the front flange 103 shown in FIG. 7 or 8, there is a front wall or face 109 from which a portion of appendix or protrusion 106 extends in a cantilever fashion; preferably, the portion of appendix or protrusion 106 extends from the front flange 103 and/or from the front face 109 in a direction substantially orthogonal thereto and/or parallel to the longitudinal axis X-X of the reel, though it may also extend radially or anyway inclined relative to said axis.

In accordance with a preferred embodiment, the appendix 106 comprises at least one pair of side walls 106a, 106b and a front wall 106c (see FIGS. 7, 8 and 15).

As will become more apparent below, the appendix 106 is configured in an advantageously original way, since it includes a series of elements, such as walls, channels, seats, etc., that require a detailed explanation. Proceeding in the right order and starting from the above-mentioned front wall 106c, coupling means 111 (more visible in FIG. 12) are advantageously provided thereon to be coupled to electric terminals 200, i.e., to respective first and second terminals 200a and 200b.

The front wall 106c can preferably also act as an abutment element for the intermediate portions 210a, 210b of the electric terminals 200a, 200b, when their coupling ends 203a, 203b are inserted in the apertures 111.

In the example shown, the front wall 106c is flat and the coupling means 111 essentially consist of seats or holes formed in the front wall 106c, where the terminals 200a, 200b can be mechanically secured by insertion and/or driving and/or fitting into a corresponding fastening end 203a, 203b.

The fastening ends 203a, 203b are so shaped or configured to ensure a stable engagement with the coupling means 111 of the reel 100; in the example shown, the fastening ends 203a, 203b are configured with a substantially T-shaped head, but they may be made differently, e.g., shaped like an arrow or a hook, or with at least partially inclined or rounded profiles, or the like.

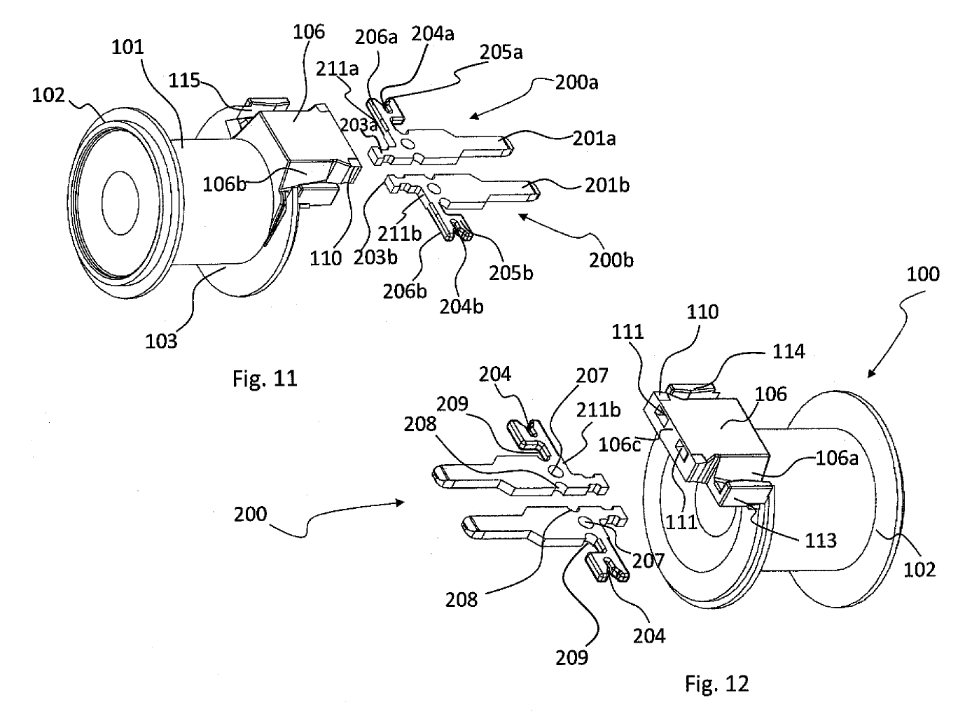

Likewise, the front wall 106b may not be flat as shown herein, but, for example, at least partially inclined or concave or convex, or may comprise distinct levels and/or cavities and protrusions, possibly in order to create at least part of a fitting or joint to be coupled to another element of the device; the front wall 106c is then preferably shaped in such a way as to also match the shape of at least some surfaces of an abutment edge 211a, 211b (FIGS. 11 and 12) of the terminals 200a, 200b.

It must also be pointed out that the terminals 200a, 200b can be fixed to the reel 100 in a different way and/or in another position, not necessarily on the appendix 106 thereof (which may even be absent or anyway have a different configuration than shown).

For example, the terminals 200a, 200b can be fixed by moulding or overmoulding the reel 100 on the respective fastening end 203a, 203b of the terminals 200a, 200b.

In this regard, it must be pointed out that the body of the reel 100 is preferably made of an electrically insulating material; to this end, it is advisable to use a polymer or a plastic material or a mouldable material, such as a thermoplastic or thermosetting material or a resin, preferably a polyamide (PA), particularly of the PA6 and/or PA66 type, or a polybutylenterephthalate (PBT) or a polyphenylensulfide (PPS), preferably with the addition of an additive or filler, such as fiberglass, e.g., in a quantity of 35%.

For brevity, the material of the reel 100 and/or of parts of the device 1 will hereafter be referred to simply as “plastic material” or “polymeric material” or “synthetic material” or “polymer” and the like, such definitions having to be understood in a broad sense to include the previously mentioned materials and/or thermosetting materials and resins, in addition to polymers and thermoplastic materials.

Referring back to the appendix 106 of the reel 100, it comprises a pair of walls or wings 113, 114 that extend laterally on opposite sides relative to a median plane of the device 1 passing through the longitudinal axis X of the reel.

In accordance with a preferred embodiment, the wings 113, 114 have a bent configuration or are inclined at different angles, so as to define, together with the side walls 106a, 106b of the appendix 106, corresponding lateral channels 115, 116 extending along the flanks of the appendix 106.

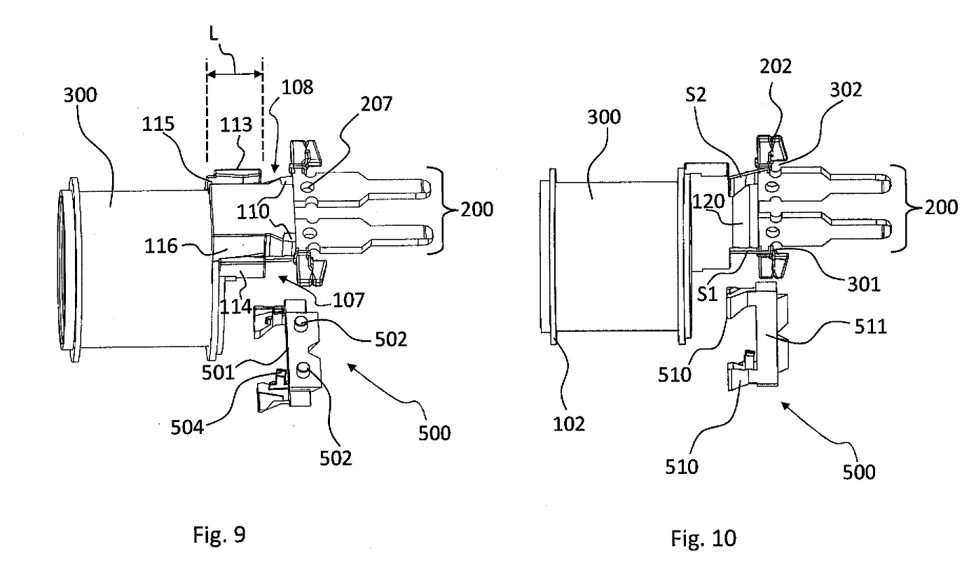

The longitudinal extension of the wings 113, 114, designated as “L” in FIG. 9, is substantially similar to that of the corresponding side walls 106a, 106b of the appendix 106, and/or at the front, i.e., at the end facing towards the terminals 200, the wings 113, 114 terminate at a shoulder or wall 118 of the appendix 106, from which a head 120 extends.

Said head 120 is a component that provides a shape coupling with at least a part of an insert 500, corresponding to the previously defined additional or auxiliary support and/or protection means or elements or inserts, which will be further described below; it must nevertheless be observed here that the head 120 of the appendix 106 is frontally delimited by the front wall 106c, where there are the holes 111 for the connection to the electric terminals 200a, 200b.

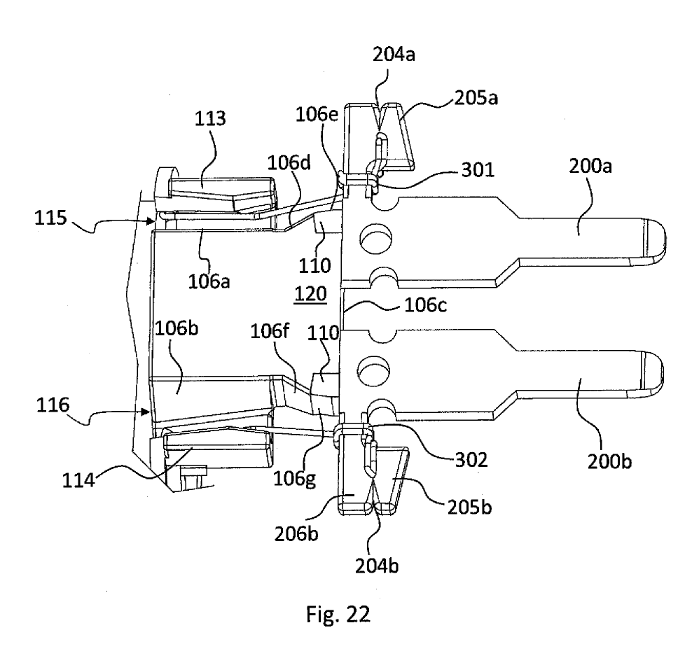

Furthermore, the head 120 of the appendix 106 comprises a series of side walls 106d, 106e, 106f, 106g, . . . 106n, which are preferably flat and oriented according to a polygonal line, as shown in FIG. 22; the function of such walls will become apparent below, but here it must be pointed out that they may also be curved or anyway not flat.

The electric terminals 200a, 200b are preferably made of an electrically conductive material; for this purpose, it is preferable to use a copper-based alloy, such as a phosphorous bronze alloy or a CuSn6 alloy, which typically comprises copper and approx. 6% tin; the electric terminals are preferably sheared from a strap and/or shaped and then coated with a tin layer.

The first and second terminals 200a, 200b comprise respective contact ends 201a and 201b, adapted to create an electric connector towards an external wiring, not shown in the drawings; said terminals 200a, 200b preferably extend parallel to the axis X of the device.

In accordance with a preferred embodiment, the electric terminals 200 have a tapered configuration towards the contact ends 201, preferably comprising or being associated with an intermediate portion 210 that is wider than the fastening ends 203 and the contact end 211.

Transversally to each intermediate portion 210a, 210b of the terminals 200a, 200b, a respective electric connection or fitting lug 202a and 202b extends, which is adapted to establish e mechanical and/or electrical fastening of the wires or ends 301 and 302 of the winding 300.

Advantageously, on each fastening or fitting lug 202a, 202b there is a respective slot 204a, 204b, which is substantially straight and transversal to the terminals 200a, 200b.

The slots 204a, 204b separate respective reliefs or flaps 205a, 205b and 206a, 206b of the corresponding lugs or fittings 202a, 202b; advantageously, the thickness of the latter or of some flaps 205, 206 is such that, as will be explained below, they can be bent or anyway deformed to allow fastening the ends of the electric wire.

The terminals 200 and/or said intermediate portion 210 further comprise seats and/or apertures and/or holes and/or notches and/or undercuts 207, 208, 209 for fastening other elements, e.g., mounted and/or overmoulded ones, as will be explained below; it should also be pointed out here, for clarity, that in this description and in the drawings the elements associated with the first terminal 200a are designated by letter “a” (e.g., 207a, 208a, 209a), while those associated with the second terminal 200b are designated by letter “b” (e.g., 207b, 208b, 209b); however, said elements are also designated, in general, by the same reference numerals with no letter.

Along the body of the reel 100, near the terminals 200a, 200b, respective clearance zones 107, 108 can be identified, which are comprised between the shoulder or wall 118 of the appendix 106 and the corresponding fitting lug 202a, 202b extending in a cantilever fashion from the intermediate portions 210a, 210b.

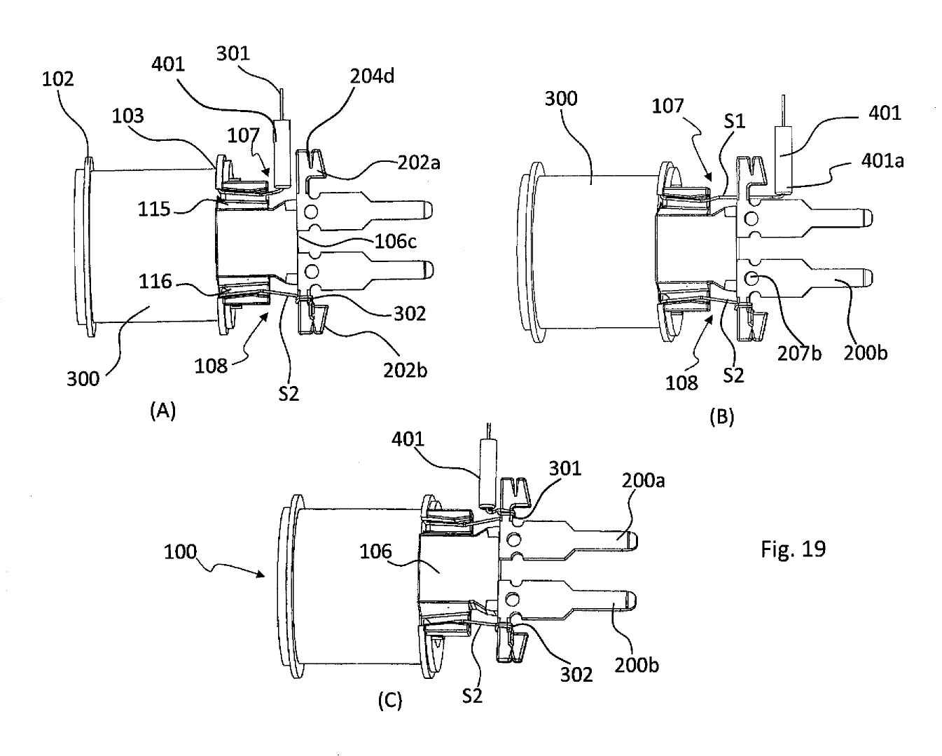

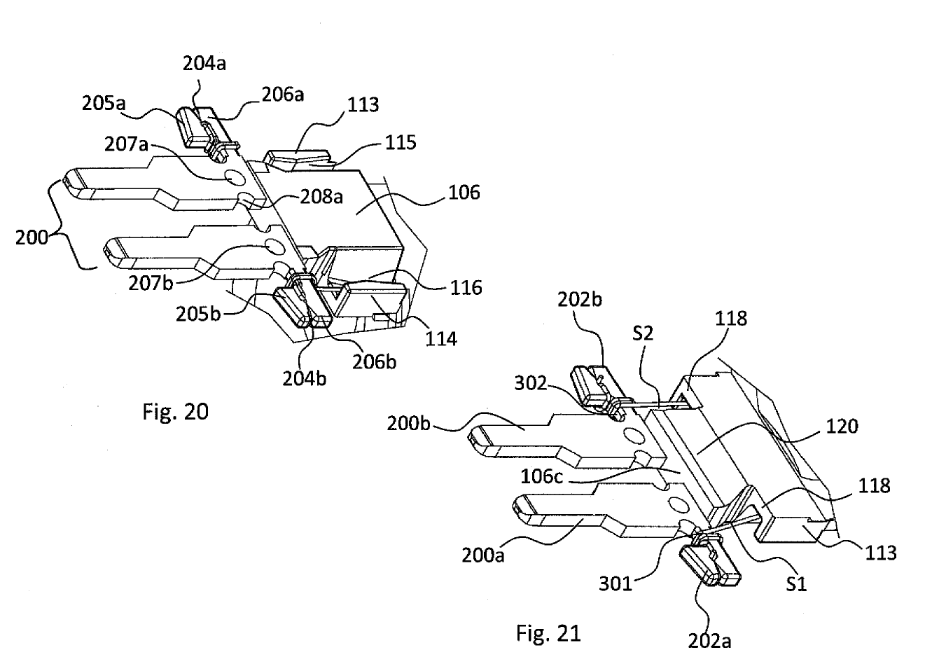

In these clearance zones 107, 108 the respective end portions 301, 302 of the electric wire 300 of the coil winding run, which are secured to the aforementioned lugs or fittings 202a, 202b; for this purpose, it is worth providing some details about how the wire 300 is applied onto the reel 100, with particular reference to FIG. 19.

There is shown the device 1 in a condition in which the wire end 302 has already been fastened to the respective fitting lug 202b and the wire 300 has already been wound on the central body 101 of the reel 100.

FIG. 19 concerns, therefore, the fastening of that end which will be defined herein, for simplicity, as first end 301 (in order to distinguish it from the other one, which will be defined as second end 302).

Note that the fastening of both ends 301 and 302 of the wire occurs in the same way; therefore, what is illustrated in FIG. 19 for the first end 301 is also applicable to the second end 302, and vice versa.

Therefore, as shown in FIG. 19, the end 302 of the wire is already in the condition in which it is fastened to a corresponding fitting lug 202b of the electric terminal 200b; for this purpose, it has been wound around the base of the lug 202b at the seat or cavity 209b, then secured by inserting its end into the slot 204b of the lug 202b and fixed by deforming the flaps 205b, 206b of the latter.

This latter step is carried out by an operator by using a tool such as pliers, or mechanically by a press.

To ensure electric contact, the end 302 of the wire is soldered to the terminal 202b, preferably by electric soldering or spot-soldering.

Still with reference to the condition of the device 1 shown in FIG. 19, after the end 302 has been fastened to the respective terminal 200b, the wire 300 is run into the channel 116 alongside the appendix 106 and wound on the central body 101 of the reel 100, in order to create the actual wire coil, and the wire is then run into the channel 115 towards the electric terminal 200a.

It is at this point that the wire end 301 is fastened to the corresponding fitting lug 202a of the electric terminal 200a: FIG. 19 specifically shows three different instants (A), (B), (C) of the part of the process for manufacturing the device of the invention.

In particular, as can be seen, in order to execute this production phase the electric wire 300 is preferably associated with a wire guide 401 and its end 301 is wound around the base of the lug 202a at the cavity or slot 209a, forming a number of loops (typically three or four, depending on the diameter of the wire 300 and the size of the cavity 209) sufficient to ensure that it will be fastened permanently.

The end 301 of the wire 300 is applied onto the lug 202a at the cavity 209a by shuttling the wire guide 401, which will make a few turns around the lug 202a by moving back and forth between the clearance zone 107 and the front zone near the tip 201a of the terminal.

As can be guessed from FIG. 19, the terminal portion 302 of the wire 300 that is present before the wire is wound on the reel 100 is secured to the fitting 202b, and is wound at the cavity 209b in the same way as previously described in regard to the fastening of the end portion 301 to the fitting 202a.

At this stage, the wire guide 401 makes a revolving motion around the fitting lug 202b, moving back and forth between the clearance zone 108 and the front zone of the terminal 200b.

As aforesaid, FIG. 19 schematically shows respective steps (A), (B), (C) of application of the end 301 of the wire onto the fastening lug or fitting 202a of the electric terminal 200a, starting from a condition in which the other end 302 of the wire has already been fastened to the respective fastening lug 202b and the wire 300 has been wound as a coil on the reel 100.

In this respect, it is worth mentioning that, as previously explained, also the end 302 of the wire 300 is applied by means of a wire guide 401, which makes a few turns around the lug 202b by passing through the clearance zone 108.

According to a preferred example, the wire guide 401 comprises a tube-shaped portion (e.g., having an outside diameter of 1.2 mm and an inside diameter of 0.6 mm for use with an electric wire having a diameter smaller than 0.3 mm), at least one end 401a of which moves in the clearance zones 107 or 108, depending on the steps of the process for manufacturing the device 1 previously described with reference to both ends 301 and 302 and to FIGS. 19(A), (B), (C), turning about the fitting lug 202a, 202b of the terminals 200a, 200b.

For this reason, the size of the clearance zones 107, 108, which are comprised between the shoulder 118 at the ends of the channels 115, 116 and the lug 202a, 202b, is such as to allow the wire guide 401 to pass as it turns to wind the ends 301, 302 of the wire on the cavity 209aa, 209b of the lug.

According to a preferred version of the device of the invention, the axial extension of said clearance zones 107, 108 relative to the axis X, i.e., the distance between the shoulder 118 of the appendix 106 of the reel 100 and the fastening or fitting lug 202a, 202b is greater than 1.3 mm, being preferably comprised between 1.5 mm and 10 mm, particularly between 1.8 and 2.2 mm.

For this purpose, the dimensions of the wings 113, 114 and/or of the side walls 106a, 106b of the appendix 106, as well as those of the respective channels 115, 116 defined by them, can be advantageously so defined as to keep said extension of the clearance zones 107, 108 within optimal values as a function of the electromagnetic device 1 to be manufactured.

Advantageously, the wings 113, 114 and the channels 115, 116 perform also the function of housing and/or partially protecting the corresponding ends S1, S2 and/or 301, 302 of the wire 300.

The ends 301, 302 of the wire 300 extend, with respect to the shoulder 118 of the appendix 106 of the reel 100, through the clearance zones 107, 108 to be then fastened to the respective fittings 202a, 202b, and therefore they have respective portions or sections S1, S2 exposed to stress caused by the flow of polymer or melted plastic injected during the moulding process.

Moreover, in addition to the above-mentioned length L, the channels 115, 116 have a width and/or a shape that allow the wire 300 to be properly positioned when it is wound on the respective fastening or fitting lug 202a, 202b of the electric terminals 200.

The width and/or the shape of the channels 115, 116 also depends, among other things, on the distance between the side wall 106a, 106b and the corresponding wing 113, 114.

In this context, it is possible to appreciate the importance of the clearance zones 107 and 108, which allow the wire guide 401 to pass around the respective fitting 202a, 202b of the terminal 200; however, this inevitably implies the presence of a section of wire, such as the sections S1, S2, that is “suspended” through the clearance zones 107, 108, i.e., between the shoulder 118 of the appendix 106 and the respective lugs or fittings 202a, 202b.

This suspended section, designated as “S1” and “S2”, is therefore exposed to the risk of breakage during the subsequent moulding or overmoulding of the plastic or polymeric material on the winding and/or the subsequent use of the product.

In fact, once the wire 300 has been applied and the ends thereof 301, 302 have been fastened, the coil is coated with electrically insulating material 400, such as a thermoplastic or thermosetting polymer, preferably by overmoulding it thereon; such material is typically injected as a fluid at a predefined pressure, and typically undergoes some dimensional shrinkage during the cooling step that follows the moulding step, said dimensional shrinkage being proportional to the thickness or mass of the moulded material.

The Applicant observed that in the clearance zones 107 and 108, i.e., in the regions where the wire guide 401 passes when the ends of the wire 300 are applied onto the fittings 202a, 202b of the electric terminals 200, a relatively high accumulation of overmoulded material 400 occurs, which envelops the tract or section S1, S2 of wire suspended in said clearance zones 107, 108.

This causes particularly high shrinkage and/or dimensional variations, especially during the production process, which directly act upon said suspended tract or section S1, S2 of wire extending between the reel and the terminal through said clearance zone 107, 108 and which will cause it to break immediately or anyway to undergo damage that may cause the wire to break and/or suffer performance reductions at a later time.

It should be considered, in fact, that even if the wire does not break immediately, strain or stress may arise which might create latent damage, i.e., partial damage, to the electric wire, e.g., by squeezing and thinning the electric wire and/or inducing strain or stress in the metal of the electric wire, or even just by damaging the insulating enamel or covering of the wire. As aforesaid, such latent damage may cause the wire to break in the long run, particularly in the case of small-gauge electric wires and/or devices used on engines and/or vehicles, i.e., devices subject to high stress, such as thermal stress and vibration.

In order to prevent such problems from arising, in the device 1 according to the invention there are additional means for protecting and/or supporting the wire 300 and/or the above-mentioned suspended tracts or sections thereof S1, S2.

In accordance with a preferred embodiment of the invention, such additional protection means comprise at least one additional insert or element 500, hereafter also referred to, for brevity, simply as “insert”, which is adapted to cover and/or protect and/or support, at least partially, those portions of electric wire 300 that are most subject to the risk of breakage or damage, such as the suspended sections S1, S2 and/or at least parts of the ends 301 and 302, etc.

In fact, the invention teaches to provide protection means adapted to achieve one or more specific and advantageous effects.

In the case of the additional insert or element 500, such effects include constituting at least one respective wall of diaphragm or septum for protecting and supporting the suspended wire tracts S1, S2.

Said diaphragm is preferably configured to be physically interposed between the suspended wire sections S1, S2 and the flow of melted and/or fluidic and/or liquid polymer or plastic material that is injected into the mould during the moulding process.

Said wall or diaphragm of the additional insert 500 is preferably configured to support the suspended sections S1, S2 of the electric wire 300, e.g., to counter a thrust exerted on said wire portion by the flow of polymeric material injected into the mould.

In a preferential configuration, the wall or diaphragm or septum provides a two-fold protection by shielding the wire tract in the clearance zones 107, 108 against a direct flow injected into the mould, which flow is diverted in the mould but then hits again on the opposite side, though indirectly, the suspended wire tracts or sections S1, S2 in the clearance zones 107, 108, where the wire is supported by the wall or diaphragm, which counters the thrust exerted by the fluid injected into the mould. Therefore, said wire shielding and supporting actions cooperate to prevent failures caused by the thrust exerted by the moulding material.

Preferably, the additional insert or element 500 also contributes to reducing the thickness of the covering material 400, in particular in said clearance zones 107, 108 where the suspended sections S1, S2 of the wire ends 301, 302 extend.

In other words, the additional element 500 is not only a diaphragm physically supporting and/or protecting the suspended wire sections S1, S2 against the flow of fluidic polymer or melted plastic during the moulding process, since it preferably also causes a reduction in the mass and/or thickness of the layer 400 of moulded polymer or plastic material along at least a part of the end portions or ends of the wire that extend in said clearance zones 107, 108 comprised between the wall or shoulder 118 of the appendix 106 of the reel 100 and the fastening lugs or fittings 202a, 202b of the electric terminals 200.

Therefore, it follows that, in these locations, the reduction in the mass and/or thickness of the overmoulded material also leads to a reduction in the shrinkage or dimensional settling of the moulded polymer or plastic material, and hence to reduced stress or strain on the wire caused by such shrinkage or settling of the moulded material.

Within the scope of this general teaching of the invention, it will thus be possible to design the insert 500 in the most appropriate manner according to the circumstances; this is an important feature of the invention.

For example, the shape and size of the coil 300, those of the electric terminals 200, as well as the number thereof, those of the appendix 106, and also other aspects, can be taken into consideration when determining the configuration of the additional element 500.

The embodiment of the additional protection and/or support element 500 shown in the annexed drawings, in particular those from 13 to 18, comprises two portions which are substantially symmetrical relative to the longitudinal axis X-X of the device, and which are adapted to be associated with and/or operate with reference to a respective part of the terminal 200a, 200b and/or end of the electric wire 301, 302 and/or suspended section S1, S2; however, two or more additional elements or inserts 500 may be used, each one associated with a respective terminal and/or a respective end 301, 302 of the electric wire and/or a respective suspended section S1, S2, such as an additional element or insert 500 of the type shown herein or the like, but divided into two parts, e.g., two symmetrical additional elements or inserts 500.

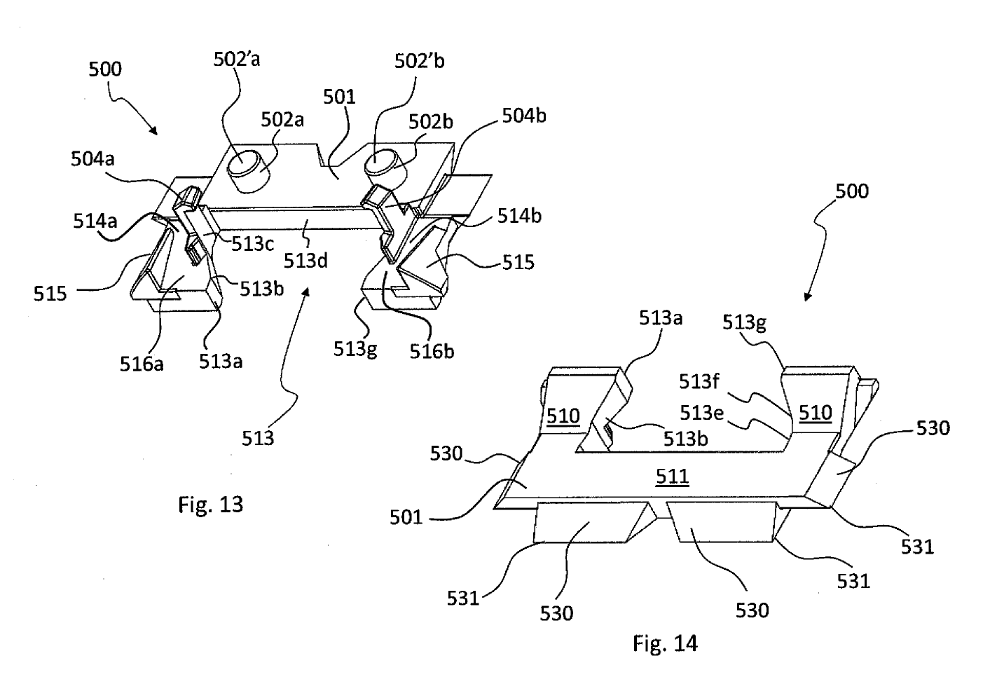

The protection insert 500 (more visible in FIGS. 13 and 14) is provided with a substantially fork-shaped body 501, wherein two portions or arms 510 extend at the end of a central portion or core 511.

The insert 500 comprises also fastening and/or coupling elements or means 502, 503, 504, 510; in this case, such means comprise protrusions or pins 502a and 502b or arms 510 that are engaged and/or coupled, preferably by interference, into respective seats or holes 207a, 207b of at least one of the terminals 200a, 200b and/or seats or rises 110 and/or walls 106d, 106e, 106f, 106g, . . . 106n (visible in FIG. 22) and/or seats or hooks defined by the appendix 106 of the reel 100.

In addition or as an alternative to this, on the body 501 of the insert 500 there may be one or more rises or joints 503, arranged centrally or interposed between the electric terminals 200a, 200b.

The rise 503 fits and/or engages, preferably by interference, into corresponding seats 208a, 208b of at least one of the terminals 200a, 200b; when these seats are not provided in the terminals 200, the rise 503 can be configured otherwise to engage with the side walls of the terminals 200.

Regardless of how the pins 502a, 502b and/or the rise 503 or other elements are embodied, in the invention the fastening and/or coupling means of the insert 500 are preferably adapted to act also as spacers or positioning elements for the electric terminals, at least during the steps that precede the moulding of the covering 400.

As can be seen, in fact, the pins 502a, 502b have a predetermined pitch, so as to preferably keep the terminals 200a, 200b apart while assembling and moulding the device 1: in this manner, higher precision and better tolerances can be achieved to improve the quality of the industrial production of the device 1.

Similar considerations also apply to the central rise 503, which, being preferably interposed between the electric terminals 200a, 200b, keeps them at a predefined distance; moreover, the rise 503 engages with the seats 208a, 208b of the electric terminals 200a, 200b and thus creates, de facto, a joint coupling that will accurately hold the same terminals parallel to each other, even while assembling and moulding the device 1.

These advantageous effects are further promoted by the fact that, preferably, the fastening means of the insert 500 comprise also coupling elements 504, e.g., teeth or hooks.

In a preferred embodiment, during the assembly operations such coupling means can bend elastically and then snap back into respective seats 110 located on the sides of the appendix 106 of the reel 100.

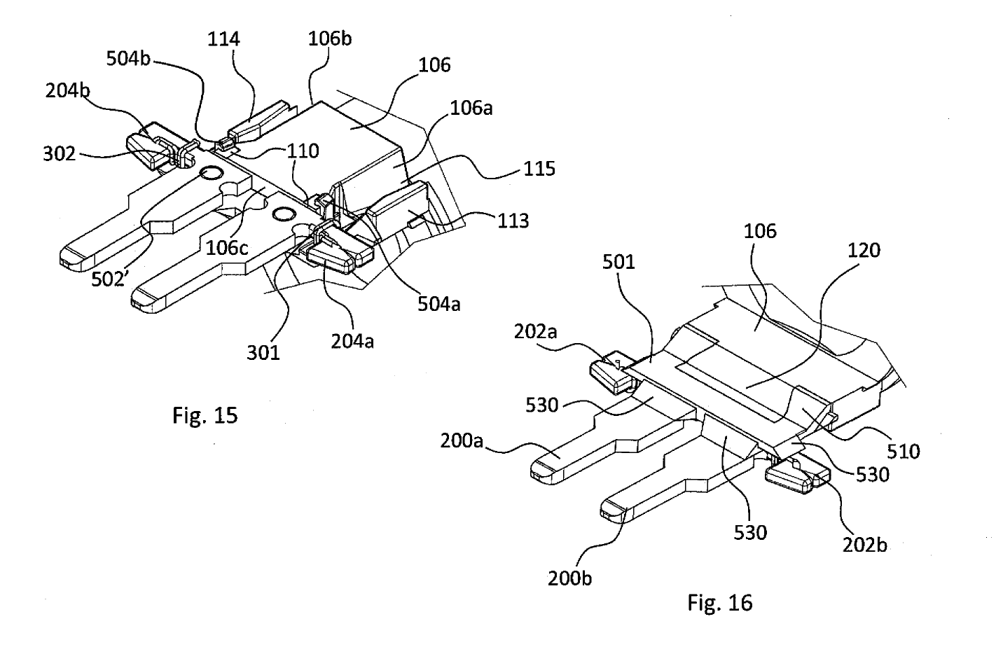

According to one advantageous embodiment, when the insert 500 is mounted to the reel 100 (as visible, for example, in FIG. 15, 16 or 17, 18), the coupling elements 504 and the seats 110 are on the side opposite to the pins 502 and/or the rise 503, with respect to the fastening lugs or fittings 202a, 202b of the electric terminals 200a, 200b, or with respect to the front wall 106c of the appendix 106, where coupling holes or seats 111 are formed.

This ensures a balanced distribution of the constraining forces that keep the insert 500 anchored to the appendix 106 of the reel 100, since it extends across said front wall 106c.

Finally, still with reference to the preferred example shown in the drawings, the body of the insert 500, with its arms 510 and central portion or core 511, circumscribes a mortise-shaped, or female-joint, seat 513 adapted to be coupled to the tenon-shaped, or male-joint, head 120 of the appendix 106 of the reel 100.

For this purpose, the tenon-shaped head 120 and the mortise-shaped seat 513 are at least partly complementary to each other and have at least some complementary or facing walls adapted to mate together, particularly by interference; preferably, the head 120 and the seat 513 and/or the arms 510 create a joint having at least some inclined walls coupled together in a “dovetail” fashion, or other joints with mating shapes having at least partially curved walls or other suitably shaped walls.

Preferably, the joint seat 513 has, at its periphery, a series of walls 513a, 513b, 513c, 513d, 513e, 513f, 513g, . . . 513n (visible in FIGS. 13 and 14) that have different orientations in accordance with a substantially polygonal line that is open at the front, where the free ends of the two arms 510 of the body of the insert 500 are positioned.

In the condition in which the insert 500 and the appendix 106 of the reel 10 are assembled together, such walls 513a-513n are juxtaposed to the walls 106c-106n of the head 120 of the appendix 106.

As better shown in FIGS. 13 and 14, in this example the walls 513a-513g are oriented symmetrically, i.e., at least the walls 513a-513c are symmetrical to the walls 513e-513g with respect to a median plane of the body of the insert 500 passing through the longitudinal axis X-X of the device 1.

Furthermore, the walls 513a, 513b e 513f, 513g, . . . 513n, i.e., those which are closer to the free ends of the arms 510 of the inserts, are inclined or converging and/or parallel; in addition, at least two of them, opposite to each other, are preferably adapted to determine a coupling interference with corresponding facing walls of the appendix 106 of the reel 100.

In general, the engagement seat 513 preferably has at least two opposite walls of the two arms 510, wherein at least one first wall 513b, 513c is opposite to at least one second wall 513e, 513f, which are inclined or diverging or converging, and/or wherein at least one wall 513a is parallel to an opposite wall 513g, particularly with respect to an axis parallel to the longitudinal axis X of the device 1.

In a preferred version, the engagement or seat 513 comprises at least two walls of the same side or arm 510 which have two different inclinations, such as the two walls 513b and 513c and/or the two walls 513e and 513f, which have different angles of inclination, e.g., relative to an axis parallel to the longitudinal axis X.

In this way, the coupling between the tenon 120 and the mortise 513 is even more stable and accurate, thus facilitating the steps of assembling and moulding the device 1.

As an alternative, the configuration or disposition of the male and female joints could be inverted. Thus, the tenon-shaped head 120 could be configured as a seat or mortise 120 to be coupled to a rise or tenon 513, still having similar profiles or walls as previously described, i.e., said seats could become rises or male joints and, vice versa, the heads or male joints could become seats or female joints.

In accordance with a preferred embodiment of the invention, the insert 500 also has some portions or surfaces 514a, 514b for bearing and/or supporting and/or protecting the respective ends or end portions 301, 302 of the electric wire, which extend in the above-mentioned clearance zones 107, 108.

The bearing and/or protection and/or support surfaces 514 extend along the intrados of the arms 510 of the insert 500, i.e., that side of said arms which, when the device 1 is in the assembled condition, faces towards the electric terminals 200.

In accordance with a preferred embodiment, said bearing and/or support portions or surfaces 514 are laterally delimited by sides 515, the latter being formed at the lateral edge of the arms 510.

Advantageously, the sides 515 are arranged substantially at right angles or rise in a direction substantially orthogonal to the bearing and/or support surfaces 514 (see FIG. 13), so that, when the insert 500 is applied onto the appendix 106 of the reel 100, the insert 500 with its sides will not interfere with the suspended end portions S1, S2 of the wire.

Furthermore, in a preferred embodiment the bearing and/or support surfaces 514a, 514b and the sides 515a, 515b are so configured as to substantially define a corresponding channel 516a, 516b for the wire 300 and/or its end sections S1, S2, 301, 302.

Advantageously, the bearing and/or support surfaces 514 and the sides 515 are so arranged as to be substantially aligned with and/or to form extensions of the wings 113, 114, so that also the respective channels 516a, 516b of the insert will be aligned with and/or will form extensions of the channels 115, 116 of the appendix 106, in particular in order to ensure longitudinal uniformity while supporting and holding the suspended sections S1, S2 of the electric wire 300 during the handling thereof for winding and/or moulding purposes.

In this context, it must be pointed out that, advantageously, also the coupling means 504 contribute to delimiting, even only partially or for a limited length, the bearing surfaces 514a, 514b on the side opposite to the sides 515.

More generally, it can be stated that some parts of the insert 500, such as said bearing and/or support and/or protection portions or surfaces 514, the sides 515, the coupling means 504a, 504b, also act as lateral containment and/or protection elements for the suspended sections S1, S2 of the wire 300 that extend in the zones 107, 108, thus being at risk of breakage or damage.

This is particularly useful for protecting the electric wire against the thrust or flow of melted or liquid polymer or plastic material while moulding the covering 400, as well as against the subsequent strain caused by shrinkage of the same material as it cools down and hardens.

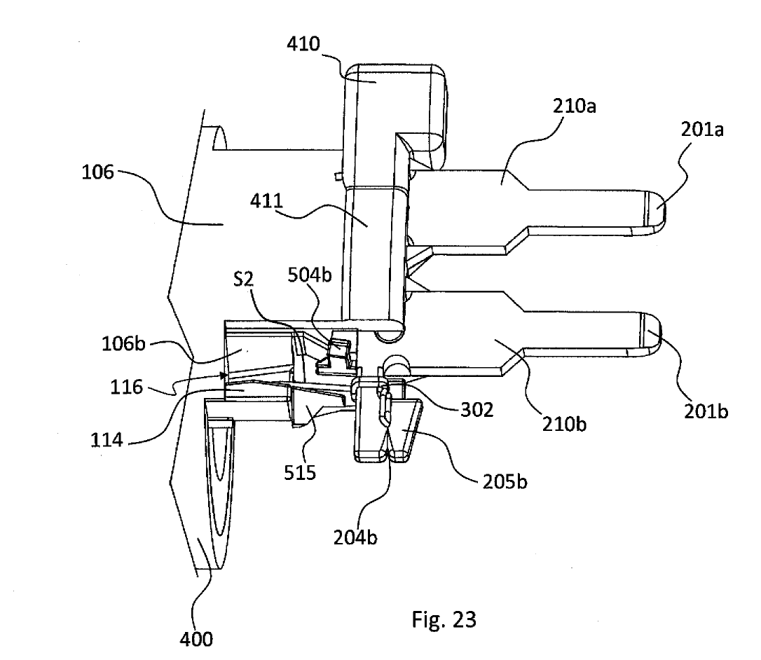

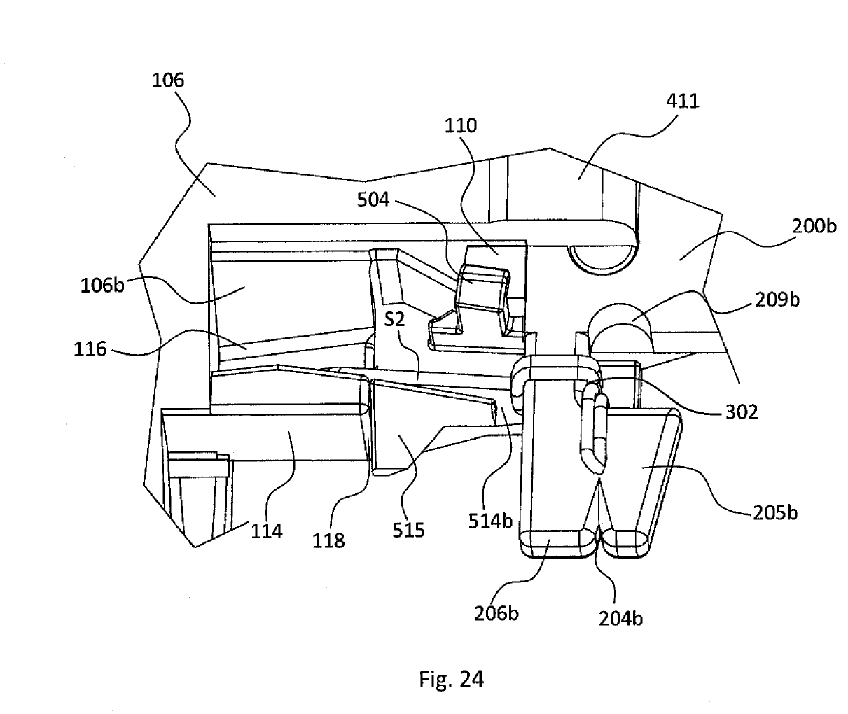

Advantageously, the effect of protecting the suspended tracts S1, S2 of the wire is amplified by the fact that, according to a preferred embodiment of the invention, the bearing and/or support and/or protection portions or zones 514 of the insert 500 are aligned with, or anyway are located along the extension line of, the guiding and/or protection and/or support channels 115, 116 that are present on the appendix 106 of the reel 100 (see details in FIGS. 23 and 24).

This provides complete and uniform protection of the ends 301, 302 of the wire 300 against any stress that may arise during the moulding and/or the subsequent shrinkage of the polymer or plastic material, particularly starting from the front flange 103 or face 109 of the reel 100, from which the appendix 106 extends, up to the electric terminals 200.

It should be taken into account that, according to the prior art described at the beginning of the present description and according to the above explanation, if no means are provided for supporting and/or protecting the suspended wire sections extending in the aforementioned clearance zones, when the polymer or thermoplastic or thermosetting material is injected where the reel assembled together with the electric wire has been previously inserted, the mass of fluidic polymer will press against the suspended tracts of the electric wire, thus increasing the risk of failure thereof.

According to the teaching of the invention, on the contrary, the means for protecting and/or supporting the electric wire 300, which in this case comprise the additional insert 500, operate in such a way as to protect and/or shield and/or support the suspended sections S1, S2; in the example shown herein, this is achieved by means of at least the surfaces 514 and, at least partly, the sides 515 and the coupling means 504, but more generally other parts of the body 501 of the insert 500 can be exploited as well.

Preferably, this effect is amplified by injecting the material into the mould in such a way that the fluidic polymer or plastic material will mostly arrive from the side of the insert 500 opposite to that where the suspended sections S1, S2 of the wire ends 301, 302 are located.

In this manner, an excessively direct or strong impact will be prevented between the fluidic mass of the moulded material and the suspended wire sections S1, S2: this will avoid, or minimize, the risk of breakage thereof due to the thrust exerted by the flow of said fluidic mass.

In this regard, it must also be pointed out that the pressure or thrust exerted by the fluidic mass is also compensated for by the protection or shielding and/or support surfaces 514 and by the sides 515 of the insert 500, preferably in combination with the channels 115, 116 and the walls 113, 114 of the appendix 106 of the reel 100, against which the suspended wire sections S1, S2 can be pushed by the fluidic polymeric mass: the action of such surfaces will support the wire, which might otherwise break.

Finally, it should be reminded that the fluidic mass injection pressure exerts a thrust also on other components of the device 1 assembled in view of the moulding process, which might get slightly displaced from the initial condition during the process.

This is the case, for example, of the electric terminals 200, which, although their ends 203 have been inserted into the holes 11 of the appendix 106, can still move because of some inevitable play.

The present invention finds a remedy to this situation in that, as aforementioned, the engagement of the insert 500 with the electric terminals 200a, 200b, provided by the pins 502 and/or the rise 503 or possibly other elements as well, will keep the electric terminals 200 accurately parallel to each other during the moulding step, thus ensuring a high-quality finished product.

In other words, it can be stated that the insert 500 can also be used as a mounting jig that will keep the assembled pieces of the device assembled together during the moulding process.

In addition to all this, it must be said that the above results also make for easier moulding, promoting the use of higher pressures and/or speeds for injecting and diffusing the fluidic polymeric material into the mould, particularly for the purpose of reducing the moulding cycle time and hence the cost of the device 1.

In accordance with a preferred embodiment, the body 501 of the insert 500 has different heights or a tapered configuration, in particular starting from the arms 510 or wings 515 up to the central core 511 and/or its anchoring tabs 530; in the assembled condition, the thickness of the insert 500 preferably decreases progressively from the front wall 109 of the reel to the free end with the front wall 106c of the appendix 106.

Preferably, said tapered configuration facilitates the fixing of the insert 500 by the fluidic polymeric mass.

Assembling an insert 500 in the front region of the appendix 106 will reduce the mass of moulded material in that region, thus also reducing said risk of damage.

According to a preferred embodiment, the shape of the arms 510 of the insert 500 has increasing thickness towards the free ends of the arms 510, in particular a thickness or shape similar to that of the appendix 106, thus providing an overall configuration of the appendix 106 with a tapered insert 500 of increasing thickness, as aforesaid.

In addition to all this, it must be said that the presence of the insert 500 of the invention, in particular of the portions or arms 510 thereof that extend in the clearance zones 107, 108, determines a reduction in the thickness or mass of the covering or overmoulding material in the region where the suspended sections S1, S2 and the ends 301, 302 of the electric wire are located: this will reduce the shrinkage of the covering and/or overmoulding material in said regions, resulting in less mechanical strain or stress on the electric wire, as well as no risk of immediate breakage or latent damage.

Finally, it must be pointed out that, according to a preferred version, the additional insert 500 also has some anchoring elements 530 acting upon the covering layer or body 400, preferably in the form of lateral and/or frontal anchoring tabs 530; as will become apparent below, a wedge-like shape is most suited for the anchoring tabs 530, even though it may be replaced by other shapes, e.g., a step-like shape or lowered-seat shape.

Preferably, the anchoring elements 530 comprise at least a part or face adapted to be covered by the covering or overmoulding material 400 and/or comprise thinner end portions 531, particularly suited to be melted, at least partially, by the covering or overmoulding material 400 with which they come in contact, so as to obtain a welding and/or a chemical bond between at least a part of the body of the additional insert 500 and at least a part of the covering layer 400 of the device 1.

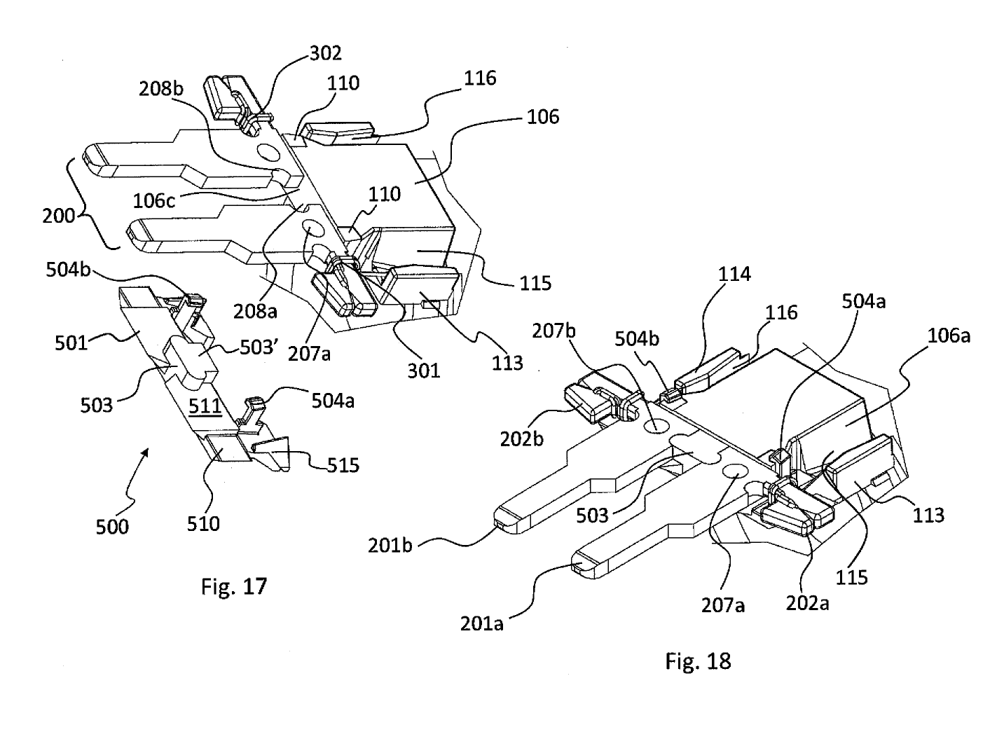

The material of the covering layer 400 envelops at least a part of the additional insert 500, possibly leaving uncovered some points thereof that may be useful for supporting and/or centering the assembly, particularly the semifinished product consisting of the reel 100 with the terminals 200, the winding 300 and the inserts 500 assembled together, in the mould 600. Preferably, the polymeric covering material 400 envelops a part of the terminals 200, penetrating also into at least some seats and/or holes and/or apertures 207, 208, 209 for improved fixing. According to an inventive aspect, the covering or overmoulding material 400 is melted and/or chemically bonded with at least a part of the fastening and/or coupling means 502, 503, 504, 510, in particular at least the ends 502′ (502′a, 502′b) of the pins 502 (502a, 502b) and/or 503′ of the rise 503 (see FIGS. 13 and 17) inserted in the seats and/or apertures 207, 208, for the purpose of improving the fixing of the insert 500 and/or terminals 200.

Through an adequate selection of the materials of the insert 500 and covering 400, of the dimensions of the parts, and of the temperatures and/or times of the overmoulding process, it is possible to cause the ends 502′ of the pins 502 and/or of the rise 503 to partially melt with the plastic material of the covering 400, thereby fixing the insert 500.

Said polymeric material of the covering 400 may also serve the purpose of forming, at least partly, the body or casing of an electric connector and/or coupling means for establishing a connection to another electric connector, such as the connector of an external electric wiring; the material of the covering layer 400 may possibly form at least a part of the body or casing of an electrovalve or an injector device.

In the light of these considerations, it is possible to explain in a simplified manner the method for industrial production of the electromagnetic device 1 according to the invention, by dividing it, for simplicity, into two main steps: a first step of assembling the various parts and winding the coil of wire 300 on the reel 100, thereby obtaining a semifinished or intermediate product, whereon a second step of overmoulding the covering of polymeric material is then carried out.

In turn, these main steps can be divided into a number of sub steps.

For example, according to a preferred method for making or assembling the device 1 of the invention, a reel 100 provided with electric terminals 200 is prepared, which can be made, for example, by inserting the terminals 200 into the reel 100, which is manufactured separately, or by overmoulding a polymeric material of the reel 100 on the terminals 200.

In the next step of the process for making the device 1, the electric wire 300 is wound on the reel 100.

To this end, as previously explained, this step is carried out by first fastening an end (301 or 302) of the electric wire 300 to a lug or fitting (202a or 202b) of a respective terminal (200a or 200b), which provides a mechanical and electrical connection for the corresponding end (301 o 302) of the winding 300.

After this, the electric wire 300 is wound on the body 101 of the reel 100 to form the main part of the electromagnetic coil, the second end of which (301 or 302) is fastened to the fitting lug (202a, 202b) of the other terminal (200a, 200b), just like the previous one.

It is worth mentioning that the order in which the ends 301, 302 are fastened can be reversed compared to FIG. 19, which should not be understood to set a specific limitation.

Furthermore, as already explained, the winding of the wire 300 and ends 301, 302 thereof is preferably accomplished by using a wire guide 401; however, the latter might not be used, or might be replaced with another equivalent tool.

Once this step is complete, a semifinished (or intermediate) product like the one shown in FIGS. 7 and 8 is obtained, whereon the additional insert 500 is then applied.

The step of assembling the additional insert 500 depends on the configuration of the insert itself and/or on the way in which it is coupled to the reel 100 and/or the appendix 106 thereof and/or at least one of the electric terminals 200a, 200b.

In the case shown in FIGS. 9 and 10, for example, the insert 500 is juxtaposed to the lugs or fittings 202a, 202b of the terminals 200a, 200b so that the pins 502a, 502b will enter the respective holes 207a, 207b in the terminals 200a, 200b, while the coupling means 504a, 504b will engage with the respective seats 110 on the appendix 106; it should however be noted that said pins 502 and/or coupling means 504 may be present in a smaller number or be replaced with other fastening means.

In the case shown in FIGS. 17, 18, wherein the rise 503 is provided as a substitute for the pins 502, when the insert 500 is juxtaposed to the terminals 200 the rise 503 will go into the corresponding seat 208, defined between the opposite seats 208a and 208b, i.e., it will enter and/or fit in between the terminals 200a and 200b.

Moreover, also the tenon portion 120 of the appendix 106 of the reel 100 and the mortise-shaped seat 513 of the insert 500 will be coupled together, thereby ensuring that the two parts are properly centered and/or joined.

As can be easily understood, the coupling between the additional insert 500 and the reel 100 is stable and accurate, so that they will remain assembled together at least during the next handling step and/or plastic/polymer overmoulding step for creating the insulating layer 400.

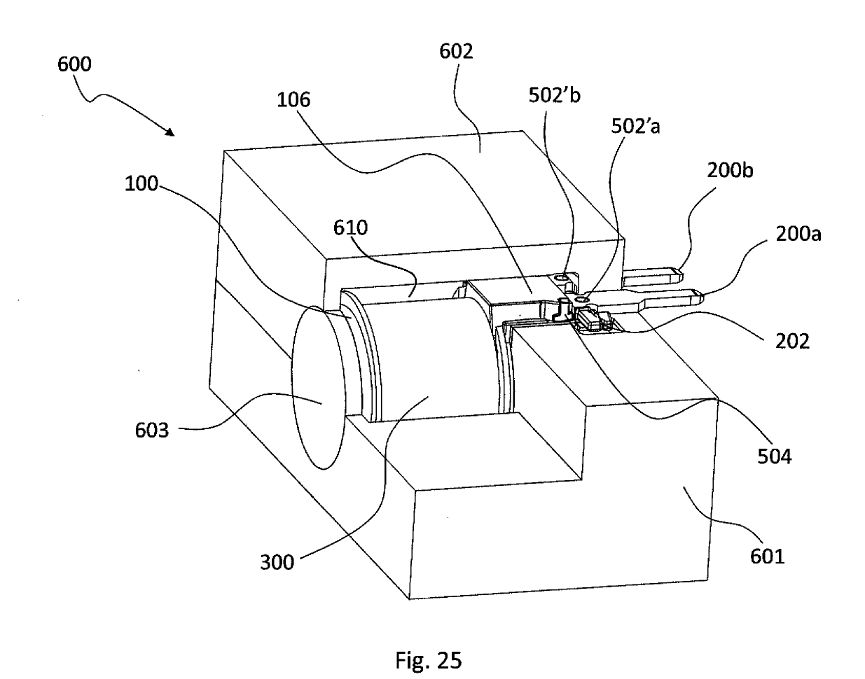

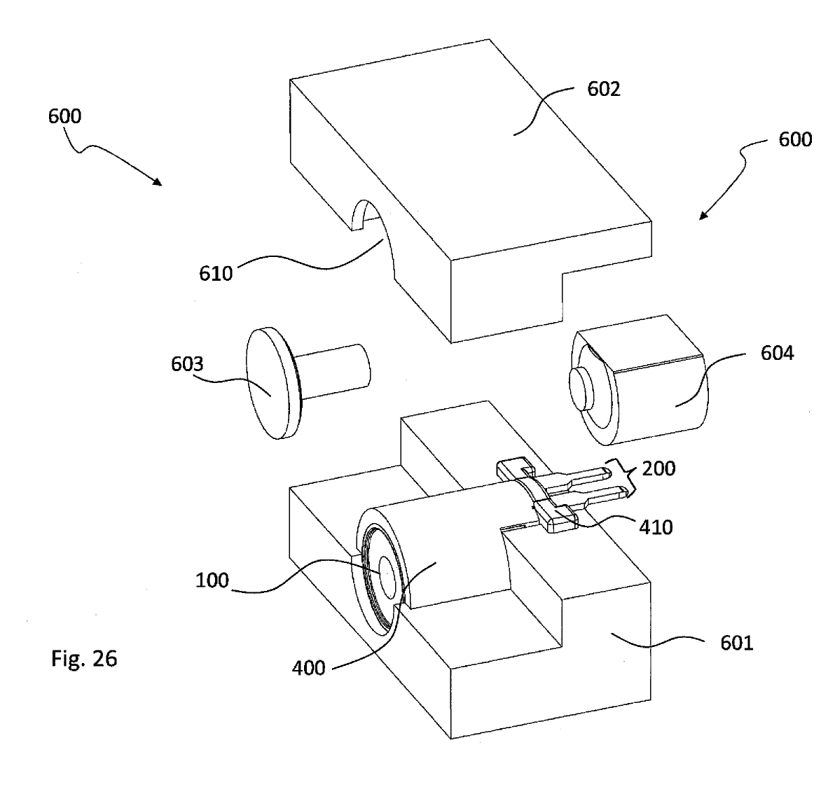

This operation is effected by placing the semifinished product into a mould, as schematically shown in FIGS. 25 and 26.

As can be seen, the first one of these drawings depicts a mould 600, with a part thereof removed to make the inside visible, which mould is intended for moulding the covering 400 over said semifinished product.

The mould 600 essentially comprises two first parts or half-moulds 601, 602, respectively a lower one and an upper one in the drawings, and two sliding second parts 603, 604 adapted to move laterally, e.g., in opposite directions along said axis X of the device 1: said second parts 603, 604 are mainly intended for forming inner parts or “undercut” parts of the device 1 and for closing the axial hole for positioning or sliding an external ferromagnetic yoke or core (not shown in the drawings) coupled to the device, while the half-moulds 601 and 602 define at least a part of the external profile of the insulating covering or casing 400.

For simplicity, a lower half-mould 601 and an upper half-mould 602 can be considered as single pieces, wherein only the upper half-mould 602 is moved in order to close and open the mould 600, while the second parts 603, 604 seal axially the moulding cavity 610 that houses the semifinished product.

The semifinished product is preferably so arranged that the ends 301, 302 of the wire and its suspended sections S1, S2 are at the top (as shown in FIG. 25), so that the fluidic mass spreading around the reel 100 will not damage them; at this stage, the insert 500 acts, as aforementioned, as a diaphragm protecting the ends 301, 302 of the wire and/or of its suspended sections S1, S2 while the fluidic mass is spreading in the mould.

After the moulding material has been injected, at least some of the parts 601, 602, 603, 604 of the mould 600, typically the half-moulds 601, and second parts 603, 604 (or other components) are opened in order to extract the finished device 1, possibly after a cooling step to allow the polymeric covering layer 400 to harden.

When the mould is opened, the axial components 603 and 604 of the mould 600 move along the longitudinal axis X of the coil, while the upper part 602 of the mould is moved away transversally to said axis X.

In accordance with a preferred embodiment of the process for making the device 1 according to the invention, the polymeric material of the insert 500 melts locally at some predetermined points to create an intimate junction with the covering 400 and become, de facto, one piece with it, resulting in clear advantages from a structural and mechanical viewpoint.

The anchoring wings or protrusions 530 promote this effect, while the mould 600 and its components 602, 604 are configured to allow the melted plastic material to penetrate around the terminals 200a, 200b, on the side thereof opposite to the insert 500.

Protection of the ends 301, 302 of the wire 300 is thus completed by an end covering 410 of the covering layer or body 400 of the device 1, formed by the overmoulded plastic material.

The end covering 410 preferably comprises a convex, or anyway curved, portion 411 following the cylindrical profile of the covering 400.

The end covering 410 is preferably configured in accordance with the presence of the additional element or insert 500, so as to protect also the lugs or fittings 202a, 202b for fastening the ends 301, 302 of the wire 300.

It must however be pointed out that the invention has been described herein with particular reference to an electromagnetic device comprising at least one wound electric wire 300, such as a solenoid or an electromagnet or an electromagnetic coil, which is preferably a part of an electromagnetic actuator of an electrovalve or an injector, in particular for apparatus or systems for injecting fuel into combustion or endothermal engines for vehicles.

Nevertheless, the same invention is also applicable to other types of devices, such as an antenna or an inductor for an electronic circuit or a winding of an electric actuator or motor or a solenoid and/or an electromagnet and/or an electromagnetic coil for different fields of application, such as the household appliance, medical, electronic or industrial industries, particularly as regards devices comprising electric wires having a diameter of less than 0.3 mm.

It is clear the devices described herein by way of example can be subjected to many variations by the man skilled in the art, without however departing from the scope of the invention. As aforementioned, further variants can be implemented by appropriately combining one or more of the features previously described with reference to different embodiments.

Furthermore, it must also be pointed out that the protection means, which in the examples taken into account herein essentially comprise the insert 500, may however be more complex.

By way of example, a dual-insert solution may be conceived, i.e., a solution comprising a supplementary insert in addition to the insert 500 described herein, e.g., arranged on the opposite side relative to the lugs 202 and the terminals 200.

Such a supplementary insert may also be different from the other insert, with specific coupling and/or engaging means for its connection to the electric terminals 200 and/or the appendix 106 of the reel and/or the insert 500, which may even differ from those described herein.

In addition, the insert 500, which in the case shown in the drawings is made as one piece, may be made as two or more pieces that can be connected to each other and/or to the electric terminals 200 and/or to the appendix 106 of the reel 100 by means of joints, couplings and the like, e.g., in accordance with the above-described teachings. In the case of two or more inserts 500 coupled together, there may be respective coupling and/or fitting elements, e.g., male-female and/or tenon-mortise pairs and/or seats and reliefs or hooks.

All of the features and variants described herein will still fall within the scope of the following claims.

1. An Electromagnetic device, preferably for electrovalves or injectors for endothermal engines, comprising at least:
a coil winding of an electric wire around a reel or the like, preferably comprising an appendix,
at least one electric terminal, secured to the reel or to an appendix thereof, comprising at least one fastening or fitting lug electrically and mechanically connected to at least an end portion of the electric wire,
wherein between the reel and said fitting or near said end portion or a section of the electric wire there is a clearance zone for the operations of winding or securing the end portion to the fitting of the electric terminal, characterized in that it comprises at least one insert or additional means for protecting and/or supporting at least a part of said end portion and/or said section of the wire, arranged at least alongside the clearance zone.

2. The device according to claim 1, wherein the protection and/or support means comprise additional or auxiliary means and/or elements and/or inserts for supporting and/or protecting and/or shielding the electric wire and/or for reducing the thickness of a material for covering the wire and/or for engagement with said at least one electric terminal.

3. The device according to claim 1, wherein the protection and/or support means comprise a body associated with or adapted to be coupled to or provided with coupling and/or engaging and/or fitting means for engagement with at least one of: the reel or a part thereof, and at least one electric terminal.

4. The device according to claim 1, comprising a covering layer or body, at least partly, on the electric winding or wire and/or the reel and/or the electric terminal, which extends at least partly up to said protection and/or support means.

5. The device according to claim 4, wherein the covering layer and the protection means are made of a respective material or polymer or plastic or other materials chemically compatible with each other.

6. The device according to claim 1, wherein said at least one terminal comprises seats and/or holes and/or slots or the like for engagement with coupling and/or engaging means of the protection and/or support means, or vice versa.

7. The device according to claim 6, wherein the coupling and/or engaging means comprise pins, rises or similar elements, which are coupled to said seats and/or holes and/or slots.

8. The device according to claim 1, wherein the protection and support means comprise at least one of: bearing and/or support surfaces, edges, channels and/or passages or the like, for the electric wire and/or end portions and/or sections thereof.

9. The device according to claim 8, wherein said at least one channel and/or passage for the electric wire and/or end portions and/or sections thereof creates an extension or extends substantially along the extension of at least one respective channel for guiding or housing the wire in the reel and/or parts thereof.

10. The device according to claim 1, wherein the protection and/or support means comprise a body in which a pair of arms extend from a core to define a joint coupling with the reel or a part thereof.

11. A method for making an electromagnetic device according to claim 1, comprising the combined steps of:
a) winding an electric wire on a reel;
b) before and/or after step a), connecting at least one end portion of the electric wire electrically and mechanically to at least one terminal associated with the reel;
d) applying or overmoulding a covering layer on the winding of electric wire and/or the reel and/or the at least one terminal, or at least a part thereof;
c), between steps b) and d), of fitting at least one insert or additional means for protecting and/or supporting the electric wire and/or at least one of its end portions and/or sections or tracts alongside a clearance zone between said at least one terminal and the reel or parts thereof,

12. The method according to claim 11, wherein step d) comprises overmoulding a polymeric or polymer material, and wherein at least a part of the protection and/or support means are made at least partly of polymeric or polymer material and are joined to the covering layer or body by melting or welding or chemical bonding of at least a part of the polymeric material of the protection and/or support means and of the covering layer.

13. An apparatus or system for injecting fuel and/or additive for internal combustion engines and/or vehicles comprising an electromagnetic device according to claim 1.

14. (canceled)

15. An electromagnetic device, preferably for apparatus or systems for injecting fuel and/or additive for internal combustion engines and/or vehicles, comprising at least one of the following:
a winding of an electric wire around a reel or the like;
an insert or means for protecting and/or supporting at least one section and/or one end portion of the electric wire,
a layer or body for covering the winding of electric wire;
electric terminals electrically and/or mechanically associated with an end portion of the electric wire;
in accordance with the above description and/or the annexed drawings.

16. The device according to claim 5, wherein the covering layer and the protection means are joined at least at one point by local melting and/or chemical bonding of the respective materials.

17. The device according to claim 10, wherein joint comprises a dovetail joint or a mortise and tenon joint.
